(19) United States
(12) Patent Application Publication
(10) Pub. No.: US 2012/0253346 A1
(43) Pub. Date: Oct. 4, 2012

(54) Three-Dimension-Extending Intramedullary Nail

(76) Inventors: Pei-Yuan Lee, Kaohsiung City (TW); Yen-Nien Chen, Kaohsiung City (TW); Wei-The Chen, Kaohsiung City (TW); Wei-Ching Wang, Kaohsiung City (TW)

(21) Appl. No.: 13/161,569

(22) Filed: Jun. 16, 2011

(30) Foreign Application Priority Data
Mar. 30, 2011 (TW) 100111025

(51) Int. Cl. A61B 17/58

(52) U.S. Cl. 606/62

(57) ABSTRACT

A three-dimension-extending intramedullary nail, which is made of memory metal, comprises a rod-shaped body, two distal ends formed at two ends of the rod-shaped body, and a plurality of bent portions on the rod-shaped body and between the two distal ends. Each of the bent portions is bent around a curve center and defines a reference plane whereon the bent portion and the curve center are disposed, and at least two of the reference planes of the bent portions are different.

### BACKGROUND OF THE INVENTION

1. Field of the Invention

The present invention relates to an intramedullary nail and, more particularly, to a three-dimension-extending intramedullary nail made of memory metal for providing a sufficient supporting stability in medical treatment of a fractured long bone.

2. Description of the Related Art

Generally, the way to treat a patient with a fractured long bone is to insert an intramedullary nail into its medullary cavity so as to bridge a defect of the fractured long bone for the bone to re-grow and recover in a short period of time.

Referring to FIGS. 1a to 1c, three insertion steps of a conventional intramedullary nail 9 disclosed by a Taiwan Patent titled as “Intramedullary Nail with Forked Head” with Publication No. 578541 is shown. The intramedullary nail 9 is in a form of a rod with a plurality of claws 91 that are formed on one end thereof and spread in different directions. Each claw 91 has a longitudinal groove 911 on a side facing away from the other claws 91, with the longitudinal groove 911 gradually deepened from a root of the claw 91 to a distal end of the claw 91. Besides, a fixing cap 92 is initially mounted to the distal ends of the claws 91 when the roots of the claws 91 are bent for the claws 91 to be drawn close to each other.

In operation, as shown in FIG. 1b, when the end of the intramedullary nail 9 with the claws 91 passes through a hole communicating with the medullary cavity of a fractured long bone and extends along the medullary cavity towards the affected part, jaws 81 of a pair of pliers 8 are movably inserted into the longitudinal grooves 911 of the claws 91 through a cut of the affected part and clip the claws 91 to prevent them from spreading. After the claws 91 are firmly clipped, the fixing cap 92 is taken off from the intramedullary nail 9, and then the intramedullary nail 9 is further pushed into the medullary cavity with the jaws 81 sliding along the longitudinal grooves 911 and gradually disengaging with the longitudinal grooves 911, so that the claws 91 can spread and stably abut against the inner surface of the fractured long bone defining the medullary cavity as the FIG. 1c showing.

However, the conventional intramedullary nail 9 still has the following drawbacks.

(A) The provided supporting stability may not be enough since the claws 91 have to be drawn close to each other for the fixing cap 92 to be mounted initially and the spread degree of the claws 91 may be lowered due to elastic fatigue of the bent roots of the claws 91.

(B) The conventional intramedullary nail 9 cannot be suitable for every kind of fractured long bones because the jaws 81 of the pair of pliers 8 have to be inserted into the longitudinal grooves 911 through the cut of the affected part at least from two opposite directions, and an affected part with a small cut impossible for the jaws 81 to clip the claws 91 in two opposite directions usually occurs.

(C) The structural design of the conventional intramedullary nail 9 is so complicated that the manufacture cost thereof is correspondingly high since each claw 91 has to form a longitudinal groove 911 with various depths. Besides, a process to form the longitudinal grooves 911 on an intramedullary nail 9 of small size may be harder and cost more than that to form the longitudinal grooves 911 on a large intramedullary nail 9.

(D) The conventional intramedullary nail 9 is weak in practicality since the intramedullary nail 9 is unbendable and can only be inserted into the medullary cavity along an axial line in alignment with a longitudinal axis of the fractured long bone, which means that the hole communicating with the medullary cavity for the conventional intramedullary nail 9 to pass through has to be formed at an axial end of the fractured long bone. However, it is not preferable to drill longitudinal axis-ends of a shin bone or a thigh bone due to the complicate structure thereof, and fractures of a shin bone or a thigh bone are common in clinical cases. Therefore, the conventional intramedullary nail 9 is not preferable in many operations for shin bones or thigh bones.

(E) Due to the constant dimensions of the conventional intramedullary nail 9, hospitals have to prepare and store intramedullary nails 9 in various sizes for patients with different builds or affected parts, and this may lead to difficulty in reserve management for the hospitals and manufactures. Besides, even though intramedullary nails 9 in various sizes are previously prepared, a desired intramedullary nail 9 perfectly fitting the medullary cavity is still seldom. In this situation, only drawing out bone medulla to deepen the medullary cavity for the intramedullary nail 9 is an effective way, and this operation may further hurt the patient.

In light of this, it is desired to improve the conventional intramedullary nail 9.

### SUMMARY OF THE INVENTION

It is therefore the primary objective of this invention to provide a three-dimension-extending intramedullary nail, which can surely abut against an inner surface of a long bone to provide an enhanced supporting stability.

Another objective of this invention is to provide a three-dimension-extending intramedullary nail, which allows a direct and uninterrupted insertion for every kind of fractured bones.

Still another object of this invention is to provide a three-dimension-extending intramedullary nail, which has simple structure and is easy to be manufactured.

Still another object of this invention is to provide a three-dimension-extending intramedullary nail, which is flexible, able to deform according to the route of a medullary cavity while being inserted into the medullary cavity, and suitable for operation with lateral insertion hole or axial insertion hole.

Still another object of this invention is to provide a three-dimension-extending intramedullary nail, which is suitable for patients with different builds or fractured bones having medullary cavities in various sizes.

Still another object of this invention is to provide a three-dimension-extending intramedullary nail, which can be firmly fixed without any conventional screws.

Still another object of this invention is to provide a three-dimension-extending intramedullary nail, which can provide a large amount of axial deformation producing a pressure toward the affected part of a long bone to pull the pieces of the fractured long bone close.

The invention discloses a three-dimension-extending intramedullary nail. The three-dimension-extending intramedullary nail comprises a rod-shaped body, two distal ends formed at two ends of the rod-shaped body, and a plurality of bent portions on the rod-shaped body and between the two distal ends. Each of the bent portions is bent around a curve center and defines a reference plane whereon the bent portion and the curve center are disposed, and at least two of the reference planes of the bent portions are different.

Furthermore, at least one of the two distal ends includes a round portion.

Furthermore, at least one of the bent portions abuts against a surface of a medullary cavity of a long bone after the intramedullary nail is inserted into the medullary cavity.

Furthermore, at least one of the bent portions is arranged in an extended portion of a medullary cavity of a long bone.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1a shows a cross-sectional view of a conventional intramedullary nail when processing a first insertion step.

FIG. 1b shows a cross-sectional view of the conventional intramedullary nail when processing a second insertion step.

FIG. 1c shows a cross-sectional view of the conventional intramedullary nail when processing a third insertion step.

FIG. 2 shows a perspective view of a three-dimension-extending intramedullary nail according to a preferred embodiment of the invention.

FIG. 3 shows a perspective view of a three-dimension-extending intramedullary nail according to the preferred embodiment of the invention when processing a first insertion step.

FIG. 4 shows a cross-sectional view of a three-dimension-extending intramedullary nail according to the preferred embodiment of the invention when processing a second insertion step.

FIG. 5 shows a cross-sectional view of a three-dimension-extending intramedullary nail according to the preferred embodiment of the invention when processing a third insertion step.

FIG. 6 shows a cross-sectional view of a three-dimension-extending intramedullary nail according to the preferred embodiment of the invention when processing a final insertion step.

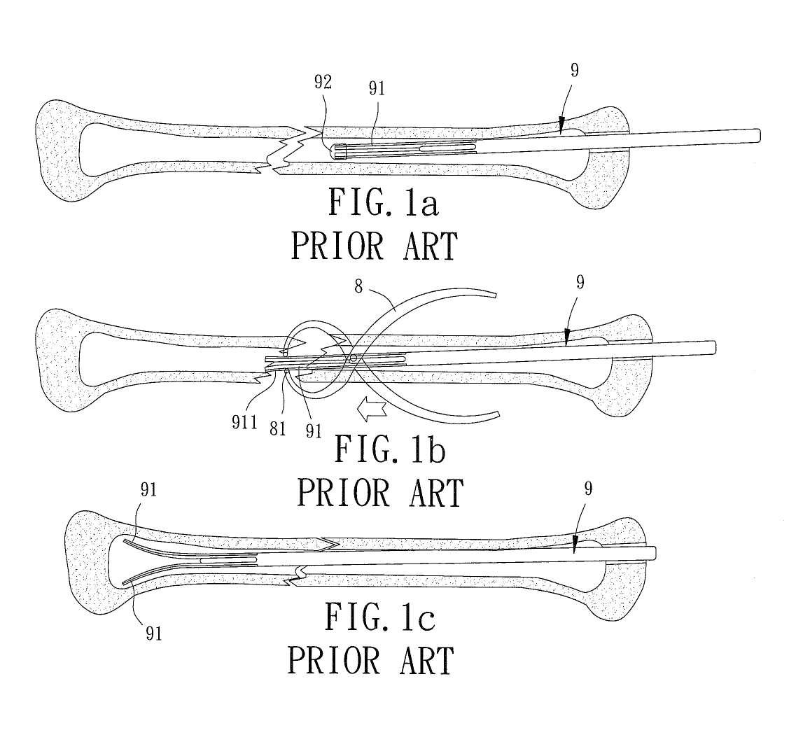

In the various figures of the drawings, the same numerals designate the same or similar parts. Furthermore, when the term “first”, “second”, “third”, “final”, “inner”, “outer” and similar terms are used hereinafter, it should be understood that these terms refer only to the structure shown in the drawings as it would appear to a person viewing the drawings, and are utilized only to facilitate describing the invention.

### DETAILED DESCRIPTION OF THE INVENTION

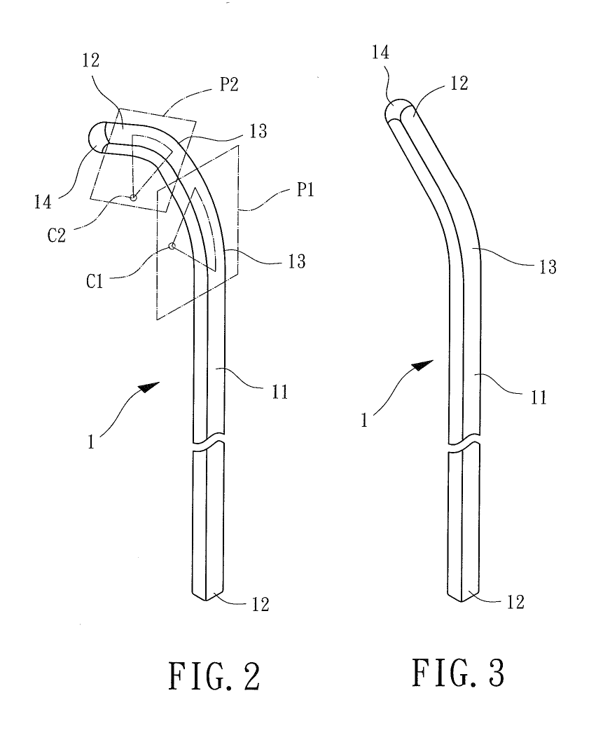

Referring to FIG. 2, a perspective view of a preferred embodiment of a three-dimension-extending intramedullary nail numbered as “1” is shown, wherein the intramedullary nail 1 includes a rod-shaped body 11, two distal ends 12 formed at two ends of the rod-shaped body 11, and a plurality of bent portions 13 on the rod-shaped body 11 and between the two distal ends 12. The shape of a cross section of the rod-shaped body 11 is a polygon or a circle, and is a quadrilateral in this embodiment for an easy manufacture. At least one of the two distal ends 12 includes a round portion 14 so as to form a round end to avoid hurting the tissues on the surface of a medullary cavity of a long bone going to receive the intramedullary nail 1. Each of the bent portions 13 is bent around a curve center while a reference plane whereon any one of the bent portions 13 and the respect curve center of the bent portion 13 are disposed is defined, and at least two of the reference planes of the bent portions 13 are different. For example, two bent portions 13 are shown in FIG. 2, one of the bent portion 13 is bent around a curve center C1 while this bent portion 13 and the curve center C1 are both disposed on a reference plane P1, and the other bent portion 13 is bent around another curve center C2 while this bent portion 13 and the curve center C2 are both disposed on another reference plane P2. With the simple structure, the manufacture of the intramedullary nail 1 is easy and thus the manufacture cost thereof is low. Besides, several kinds of the intramedullary nail 1 with different dimensions of their cross sections can be made for various long bones.

Specifically, the intramedullary nail 1 is made of implantable memory metal and preferably of a nickel-titanium alloy. The primary physical property of objects made of memory metal lies in that these objects are flexible and their shapes can be changed at a temperature lower than a transformation temperature of the memory metal and can recover to the original automatically when the temperature thereof is risen back to or higher than the transformation temperature.

Please refer to FIG. 3. In use of the present three-dimension-extending intramedullary nail, a medical operator may obtain the necessary information of the support instrument of the present invention such as the route and caliber of the medullary cavity by the x-ray of the long bone having the medullary cavity, and decides which kind of the intramedullary nail 1 is suitable according to the obtained information so that the maximum outer circumference of the intramedullary nail 1 can match with the minimum circumference of the medullary cavity of the long bone. After an intramedullary nail 1 is selected, the medical operator initially changes the shape of the intramedullary nail 1 at a temperature lower than the transformation temperature into a shape convenient for insertion, such as straightening most of the plural bent portions 13.

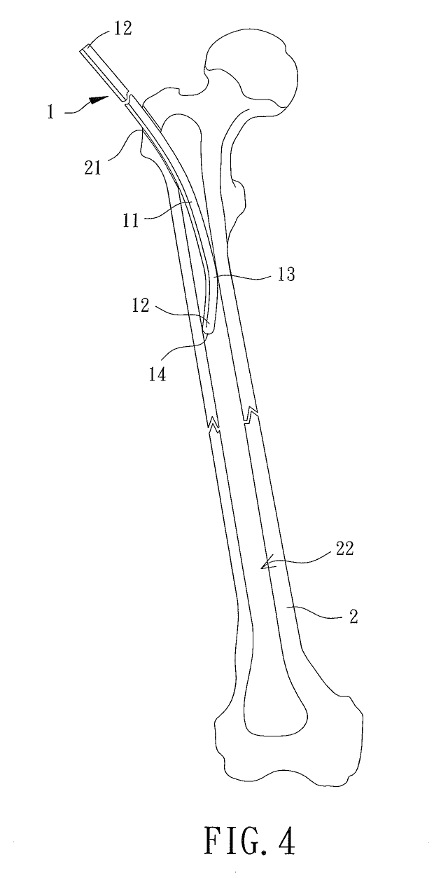

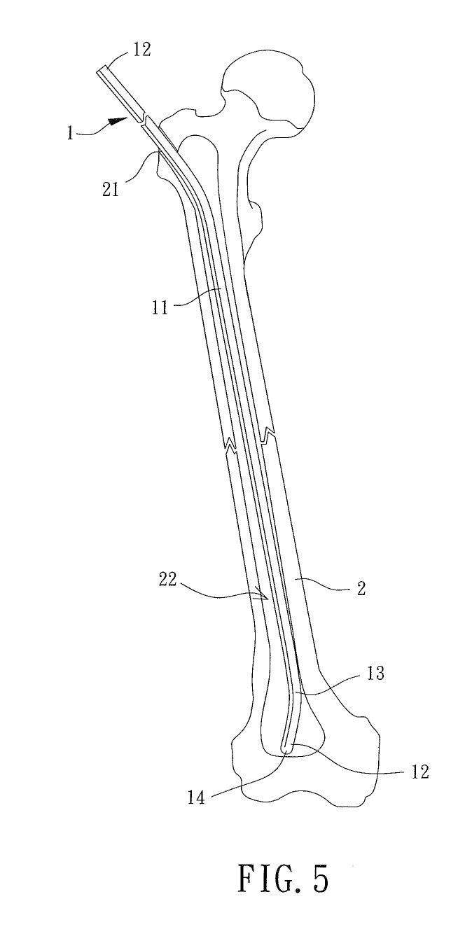

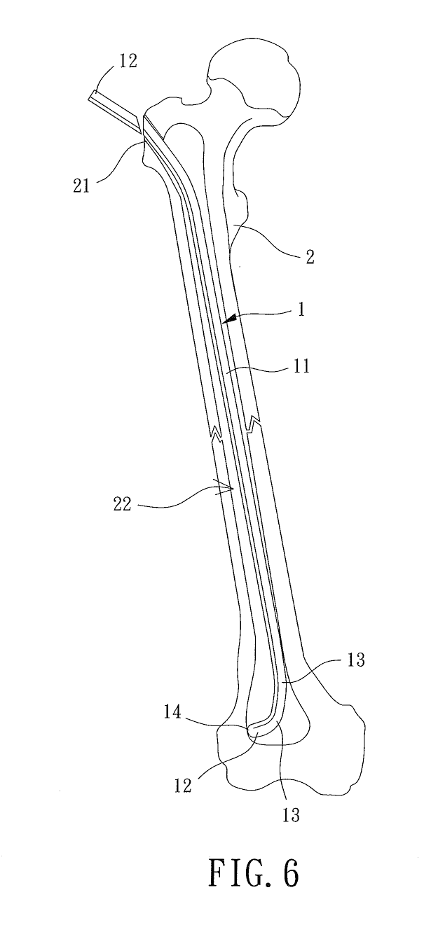

Please refer to FIGS. 4 to 6, which illustrate cross-sectional views of the intramedullary nail 1 and a long bone 2 receiving the intramedullary nail 1 in operation. The medical operator can let the round portion 14 of the intramedullary nail 1 insert into a medullary cavity 22 of the long bone 2 through a hole 21 communicating with the medullary cavity 22 as shown in FIG. 4, and keep inserting the intramedullary nail 1 into the medullary cavity 22 until the round portion 14 abuts against an end of the medullary cavity 22 away from the hole 21 as shown in FIG. 5. Preferably, at least one of the bent portions 13 is arranged in an extended portion of the medullary cavity 22. During insertion of the intramedullary nail 1, because the rod-shaped body 11 can be slightly bent to match the route of the medullary cavity 22 due to the flexibility of memory metal, the intramedullary nail 1 can be smoothly inserted into the medullary cavity 22 even if the intramedullary nail 1 did not be initially straighten to a perfect shape for insertion. Besides, the flexibility of the intramedullary nail 1 can also prevent the tissues on the inner surface of the long bone 2 from hurt. Accordingly, the intramedullary nail 1 is suitable for every kind of fractured long bones since it allows a direct and uninterrupted insertion from the hole 21 to the end of the medullary cavity 22 away from the hole 21 and no operation step has to be done through the affected part of the fractured long bone.

Furthermore, with the flexible intramedullary nail 1, operation through a lateral insertion hole taken as the hole 21 for a shin bone or a thigh bone is achievable, and this may lower the hurt to the complicate end structures of the shin bone or thigh bone, ease the difficulty of operation, and shorten the recovery time period of the patient.

Referring to FIG. 6, the temperature of the intramedullary nail 1 is then risen by the temperature of the long bone 2 after the intramedullary nail 1 is inserted into the medullary cavity 22, and thus the bent portions 13 recover to arc shapes automatically. The intramedullary nail 1 may extend in three dimensions since at least two of the reference planes of the bent portions 13 are different. Particularly, because at least one of the bent portions 13 is arranged in the extended portion of the medullary cavity 22, the intramedullary nail 1 can provide a large amount of axial deformation when the at least one bent portion 13 recovers its original shape. When all the plural bent portions 13 recover their original shape, at least one of them abuts against the inner surface of the long bone 2, and it is preferable to let a plurality of the bent portions 13 abut against the inner surface of the long bone 2 in order to provide a large supporting stability and fixing efficiency. Moreover, the axial deformation of the intramedullary nail 1 can produce a pressure toward the affected part of the long bone 2 to pull the pieces of the fractured long bone 2 close, so that time period for the long bone 2 to re-grow and recover is short.

Finally, with a conventional cutting device, a part of the intramedullary nail 1 protruding from the long bone 2 is cut off and thus the operation steps are finished. By the cut intramedullary nail 1, the difficulty in reserve management for the hospitals and manufactures may be lowered since only intramedullary nails 1 with sufficient and identical length are used, and an intramedullary nail perfect for the medullary cavity 22 of the long bone 2 is provided because the final length of the intramedullary nail 1 is decided according to the actual situation in operation. Therefore, the intramedullary nail 1 of the present invention can not only improve the convenience of operation but also shorten the operation time period, as well as provide a better help in recovering efficiency than a conventional intramedullary nail did.

In sum, with the bent portion 13 for the intramedullary nail 1 to have arcs extending on different reference planes after the intramedullary nail 1 is inserted into the medullary cavity 22, the three-dimension-extending intramedullary nail of the present invention can surely abut against the inner surface of the long bone 2 to enhance the supporting stability of the intramedullary nail 1. Moreover, manufacture cost of the intramedullary nail 1 is low since no process steps for forming any complex structure such as groove or hole is necessary. Besides, the operation object of the three-dimension-extending intramedullary nail of the present invention can be not only humans but also animals.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A three-dimension-extending intramedullary nail, which is made of memory metal, comprising:
a rod-shaped body and two distal ends formed at two ends of the rod-shaped body; and
a plurality of bent portions on the rod-shaped body and between the two distal ends, wherein each of the bent portions is bent around a curve center and defines a reference plane whereon the bent portion and the curve center are disposed, and at least two of the reference planes of the bent portions are different.

2. The three-dimension-extending intramedullary nail as claimed in claim 1, wherein at least one of the two distal ends includes a round portion.

3. The three-dimension-extending intramedullary nail as claimed in claim 1, wherein at least one of the bent portions abuts against a surface of a medullary cavity of a long bone after the intramedullary nail is inserted into the medullary cavity.

4. The three-dimension-extending intramedullary nail as claimed in claim 1, wherein at least one of the bent portions is arranged in an extended portion of a medullary cavity of a long bone.

5. The three-dimension-extending intramedullary nail as claimed in claim 3, wherein at least one of the bent portions is arranged in an extended portion of the medullary cavity.
